(19) United States
(12) Patent Application Publication
(10) Pub. No.: US 2025/0213156 A1
(43) Pub. Date: Jul. 3, 2025

(54) ORGANIZATIONAL WELLBEING

(71) Applicant: FRAMERY OY, Tampere (FI)

(72) Inventors: Tomi NOKELAINEN, Tampere (FI); Annika VÄNSKÄ, Tampere (FI); Johanna HORSTIA, Tampere (FI); Samu HÄLLFORS, Tampere (FI)

(73) Assignee: FRAMERY OY, Tampere (FI)

(21) Appl. No.: 18/850,737

(22) PCT Filed: Apr. 18, 2023
(86) PCT No.: PCT/FI2023/050217

§ 371 Date: Sep. 25, 2024

(30) Foreign Application Priority Data
Apr. 29, 2022 (FI) 20225368

(51) Int. Cl. A61B 5/16; A61B 5/11; E04H 1/12; G10K 11/16

(52) U.S. Cl. CPC A61B 5/165; E04H 1/125; A61B 5/1102; G10K 11/16

(57) ABSTRACT

A method of measuring organizational wellbeing, including gathering physiological data of a plurality of unidentified persons from at least one non-wearable sensor within an organization, and detecting events or trends indicative of organizational wellbeing from the gathered data.

### FIELD

The present disclosure generally relates to measuring organizational wellbeing.

### BACKGROUND

This section illustrates useful background information without admission of any technique described herein representative of the state of the art.

Organizational wellbeing has conventionally been measured with periodically administered surveys or questionnaires. More recently, organizational wellbeing has been measured by push-button polling devices at e.g. workplace corridors or doorways. As a more person-specific alternative, employees have been provided with personal heart rate variability (HRV) sensors which the employees attach to their bodies (i.e. body-attached electrodes) and can wear day and night to gather HRV data. Based on HRV data extracted from the sensors, person-specific scores of stress, sleep quality, and health effects of physical activity has been produced.

In recent years the stress in workplaces has been increasingly under discussion, and there is a demand for further tools for measuring organizational wellbeing.

### SUMMARY

It is an object of certain embodiments of the invention to provide a novel and inventive way of measuring organizational wellbeing.

Office pods, such as soundproof conference or phone booths, are used in modern furnishing of workplaces as well as public spaces. Such pods are often used for working, telephone calls and video conferencing.

Now it has been discovered that office pod(s) within an organization can be used to measure organizational wellbeing.

According to a first example aspect of the invention there is provided a method of measuring organizational wellbeing, comprising:
gathering physiological data of a plurality of unidentified persons from at least one non-wearable sensor within an organization; and
detecting events or trends indicative of organizational wellbeing from the gathered data.

In certain embodiments, said gathering physiological data comprises obtaining (or measuring) raw sensor data and converting it to usable information (physiological data).

In certain embodiments, said gathering physiological data comprises identifying or extracting physiological phenomenon or phenomena from the raw sensor data. In certain embodiments, such a physiological phenomenon is heart rate. In certain embodiments, such a physiological phenomenon is heart rate variability (HRV). In certain embodiments, such a physiological phenomenon is respiration rate. In certain embodiments, such a physiological phenomenon is yawning. In certain embodiments, such a physiological phenomenon is laughter. In certain embodiments, different physiological phenomena are identified or extracted which may include more than one of the preceding phenomena.

In certain embodiments, the method comprises gathering anonymous physiological data that is aggregated, e.g., processed in a statistical format (e.g. averaged). Accordingly, in certain embodiments, the method comprises processing the gathered data as aggregated anonymous data (the data being not linked to individual users (employees) of the organization).

In certain embodiments, said physiological data are indicative of physiological response(s) to human emotional state(s). In certain embodiments, said physiological data are indicative of human physical condition.

In certain embodiments, said gathering physiological data comprises: gathering physiological data of a plurality of unidentified persons from at least one non-wearable sensor in conjunction with a meeting space.

In certain embodiments, said at least one non-wearable sensor is selected from a group consisting of sensors that detect movement, such as a pressure sensor, a radar, and/or an acceleration sensor.

In certain embodiments, said at least one non-wearable sensor is integrated into a seat within the meeting space.

In certain embodiments, the meeting space is a soundproof booth.

In certain embodiments, said gathering physiological data comprises obtaining a ballistocardiographic (BCG) signal.

In certain embodiments, said gathering physiological data comprises identifying laughter. Further, in certain embodiments, said detecting events and trends comprises detecting or monitoring organizational happiness.

In certain embodiments, said gathering physiological data comprises identifying an indication of physiological stress.

In certain embodiments, said detecting events or trends comprises comparing gathered physiological data at an organizational level between different points in time. Herein comparing “at an organizational level” means comparing statistically (by averaging or by another statistical method) obtained values representing a plurality of individuals within the organization, rather than comparing physiological data of an individual person with their preceding data. In certain embodiments, said detecting events or trends comprises comparing statistically processed (such as averaged) gathered (anonymous) physiological data over time.

Further, in certain embodiments, said detecting events or trends comprises detecting an instant statistical change (event) and/or a long-term statistical change (trend) in gathered physiological data (at an organizational level).

In certain embodiments, detecting the events and trends comprises comparing anonymous physiological data of a first set of persons (representing the population of the organization at a given time) with anonymous physiological data of a second set of persons (representing the population of the organization at another time) over time (or between time instants), wherein the first and second sets of persons differ from each other.

In certain embodiments, the method comprises providing access to the detected events or trends (for further actions, e.g., a corrective action).

In certain embodiments, said detecting events or trends comprises:
monitoring physiological data before and after an organizational incident; and
reporting a detected statistical change in said physiological data (at an organizational level) after the incident.

According to a second example aspect of the invention there is provided an apparatus, comprising:
at least one processor; and
at least one memory including computer program code, the at least one memory and the computer program code being configured, with the at least one processor, to cause the apparatus to perform the method of the first aspect or any of its embodiments.

According to a third example aspect of the invention there is provided a soundproof booth (office pod or similar) for use in the method of the first aspect or any of its embodiments, comprising:
a non-wearable sensor for gathering physiological data of a plurality of unidentified persons within the booth for an apparatus that gathers physiological data of a plurality of unidentified persons from at least one non-wearable sensor within an organization and detects events or trends indicative of organizational wellbeing from the gathered data.

According to a fourth example aspect of the invention there is provided a computer program comprising computer executable program code which when executed by at least one processor causes an apparatus to perform the method of the first aspect or any of its embodiments.

According to a fifth example aspect of the invention there is provided a computer program product comprising a non-transitory computer readable medium having the computer program of the fourth example aspect stored thereon.

According to a sixth example aspect there is provided an apparatus comprising means for performing the method of the first aspect or any related embodiment.

Any foregoing memory medium may comprise a digital data storage such as a data disc or diskette, optical storage, magnetic storage, holographic storage, opto-magnetic storage, phase-change memory, resistive random access memory, magnetic random access memory, solid-electrolyte memory, ferroelectric random access memory, organic memory or polymer memory. The memory medium may be formed into a device without other substantial functions than storing memory or it may be formed as part of a device with other functions, including but not limited to a memory of a computer, a chip set, and a sub assembly of an electronic device.

Different non-binding example aspects and embodiments have been illustrated in the foregoing. The embodiments in the foregoing are used merely to explain selected aspects or steps that may be utilized in different implementations. Some embodiments and features may be presented only with reference to certain example aspects. It should be appreciated that corresponding embodiments and features apply to other example aspects as well. In particular, the embodiments and features described in the context of the first aspect are applicable to each further aspect, and vice versa. Any appropriate combinations of the embodiments may be formed. Any apparatus and/or methods in the description and/or figures not covered by the claims are examples useful for understanding the invention.

### BRIEF DESCRIPTION OF THE FIGURES

Some example embodiments will be described with reference to the accompanying figures, in which:

FIG. 1 shows an office pod in accordance with certain embodiments;

FIG. 2 shows an example framework in accordance with certain embodiments;

FIGS. 3a-3b show certain details of a seat in accordance with certain embodiments;

FIG. 4 shows a schematic block diagram of an apparatus in accordance with certain embodiments;

FIG. 5 shows a flow chart of a method in accordance with certain embodiments;

FIG. 6 shows a flow chart of a method in accordance with certain other embodiments; and

FIGS. 7a-7d show identification of certain features in a ballistocardiographic signal in accordance with certain embodiments.

### DETAILED DESCRIPTION

In the following description, like reference signs denote like elements or steps.

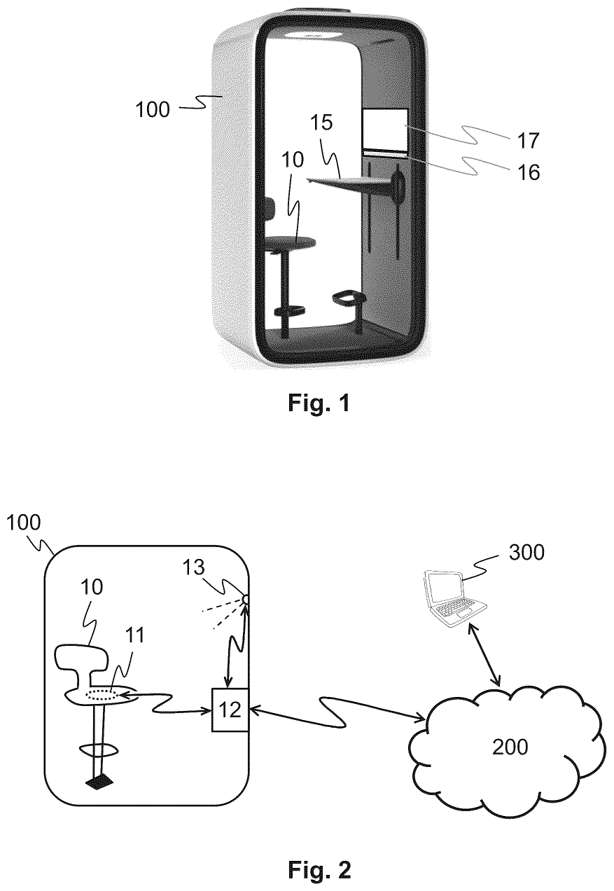

Reference is made to the FIGS. 1-7 with the following numerals and denotations:
10 Seat
10a Seat bottom
10b Seat cover
10c Back rest
11 Pressure sensor
12 Communication unit
13, 13′ Second sensor
16 Audio system
17 Display
100 Office pod
200 Cloud
300 Terminal device
40 Apparatus
41 Processor
42 Memory
43 Work memory
44 User interface
45 Communication interface
46 Non-volatile memory
47 Program code
48 Data
51-52 Method steps
61-63 Method steps
71, 71′ Ballistocardiographic signal
73 Respiratory curve
75 Short laugh
76 Big laugh
77 Very short laugh
78 Speaking aloud

FIG. 1 shows an office pod 100 in accordance with certain embodiments. As an example, the pod 100 encloses a soundproof space for working, telephone calls and video conferencing. The pod comprises a seat 10 for the user to sit on, and optionally a work surface 15 attached to a wall. In certain embodiments, the pod 100 provides wireless connectivity for communication devices that users carry in into the pod 100. Further, a user can in many cases use an audio system (including one or more speakers and/or one or more microphones) 16 of the pod 100 with their communication device. Further, the pod 10 may contain a display 17 for the use of the user. In certain embodiments, the display 17 is considered to comprise at least one of the following displays: a display for the user to interact with the pod 100 systems, and a display for the use of the user's communication device. These displays may be incorporated into the one and the same display or they may be implemented as separate displays.

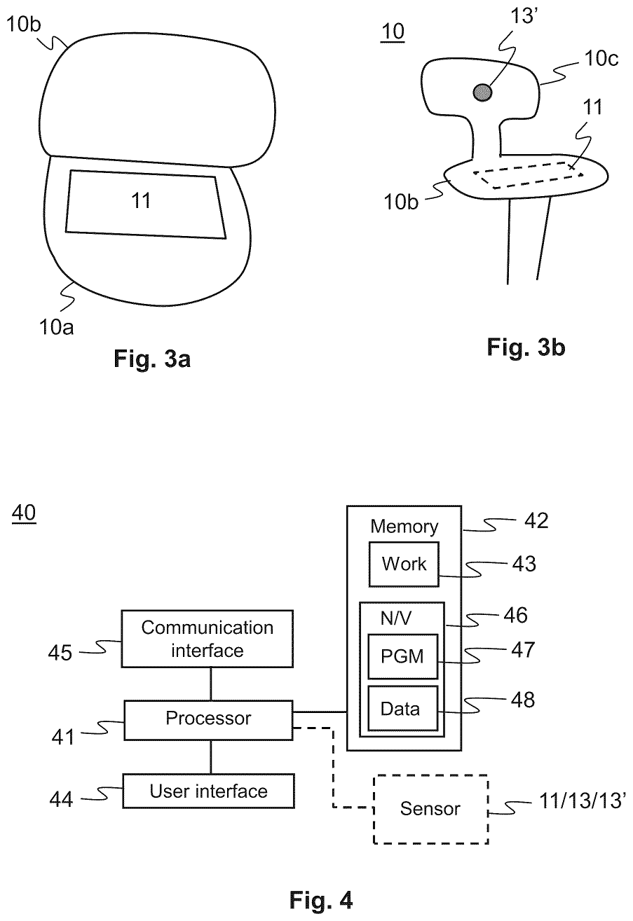

FIG. 2 shows an example framework in accordance with certain embodiments for measuring organizational wellbeing. The office pod 100 comprises a (or at least one) non-wearable sensor 11 for gathering physiological data of a plurality of unidentified persons. In an embodiment, the sensor 11 is integrated (or embedded) into the seat 10. In such an embodiment, the sensor 11 may be a sensor that detects movement, such as a pressure sensor, and/or an acceleration sensor. In certain embodiments, the sensor 11 is in the form of a pressure sensitive foil. In certain embodiments, the sensor 11 comprises a piezoelectric layer, and the movement detection is based on a piezoelectric effect. The sensor 11 may be positioned in between a seat bottom 10a and a seat cover 10b as shown in FIG. 3a (the seat cover 10b is shown in a raised position in FIG. 3 to reveal the sensor 11 underneath it). In other embodiments, the sensor is a radar, in particular a millimeter wave (mmWave) radar shown by reference numeral 13. This “second” sensor 13 provides for an alternative sensor or an additional sensor to the sensor 11. In certain embodiments, the “second” sensor 13 is attached to a wall of the pod 100, whereas in certain other embodiments, as shown in FIG. 3b, a second sensor 13′ (which may be either of the type of the sensor 11 or a radar) is integrated (or embedded) into a back rest 10c of the seat 10.

The purpose of the sensor(s) is to measure physiological data of a person residing within the pod 100. The sensor(s) measure bodily movements of the sitter (user of the pod). The bodily movements, inter alia, include movements caused by heartbeats, respiration, and also bodily jiggling due to laughing. The seat-embedded sensor 11 measures bodily movements of the sitter (user of the pod) as transmitted to the seat 10 through the buttocks of the sitter. Similarly, the sensor 13′ measures bodily movements of the sitter as transmitted to the back rest 10c through the back of the sitter. In the radar implementation of the sensor 13′ (and that of sensor 13) the sensor measures bodily movements through radar waves.

Positioning a sensor at the seat 10 underneath the sitter has the advantage that constant pressure caused by the sitter is larger and thus the disturbance caused by non-desired disturbing bodily movements (that do not relate to desired bodily movements that reflect desired physiological data) is relatively smaller. Positioning a sensor in the back rest 10c has the advantage of a closer distance to heart and lungs.

Each particular embodiment may involve one on more sensors, and the present disclosure is not limited to a specific sensor technology.

The used at least one non-wearable sensor provides a measurement signal (raw sensor signal or data). In certain embodiments, the signal provided by the at least one non-wearable sensor is a ballistocardiographic (BCG) signal. However, in other embodiments, another signal reflecting bodily movements is used.

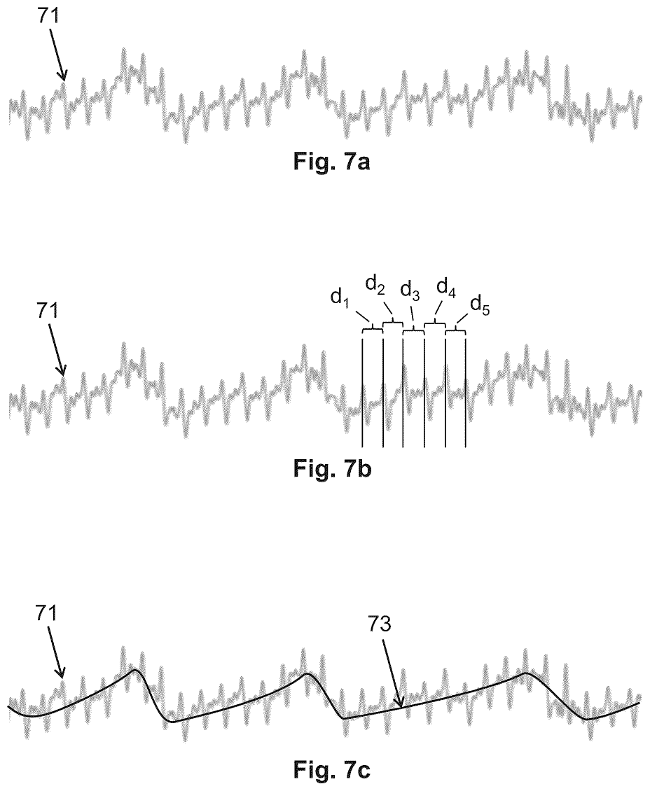

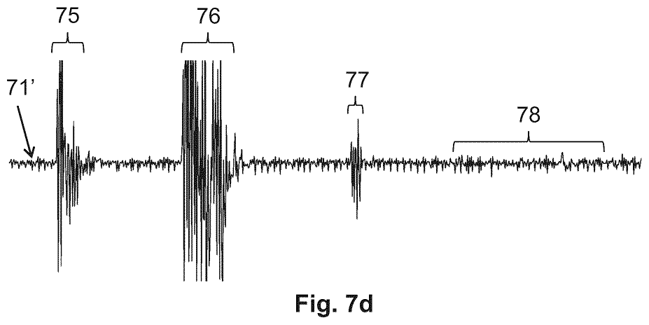

The provided raw sensor signal (that reflects bodily movements) is converted into usable information (physiological data). In certain embodiments, the physiological data are indicative of physiological response(s) to human emotional state(s) or indicative of human physical condition. Examples of information content contained by the physiological data are heart rate variability (indicator of negative bodily stress) and laughter (indicator of “positive” stress, or organizational happiness and joy). The presented method also enables detecting certain physical conditions, such as abnormal functioning of the heart (“cardiac arrhythmia”). The conversion into usable information in certain embodiments involves identification and extraction of the desired information from the raw sensor signal by an appropriate algorithm. This is illustrated later in this description with reference to FIGS. 7a-7d.

The physiological data is gathered into a memory for analysis. In certain embodiments, the data is gathered generally into a cloud service 200 which can be accessed and administrated e.g. by a terminal device 300.

The users (employees) that use pod 100 are not identified for the purpose of the measurements. Accordingly, the gathered data thus contains physiological data of a plurality of unidentified persons within the organization.

Depending on the embodiment, the raw sensor signal or data measured by the sensor 11/13/13′ is initially processed (converted into usable information) at the sensor 11/13/13′. The sensor 11/13/13′ comprises a communication interface, preferably a mobile communication interface, for transmitting the measurement data to a pod communication unit 12. The pod communication unit 12 may process the measurement data further (or instead of the initial processing at the sensor 11/13/13′). The pod communication unit 12 then transmits the processed measurement data (or unprocessed measurement data in certain embodiments) into the cloud service 200. The cloud service 200 receives the measurement data measured at the pod 100. The data transmission is preferably carried out wirelessly. In other embodiments, the sensor 11/13/13′ may transmit the data directly to the cloud service 200 e.g. over a Wi-Fi network or via a mobile data service. In yet other embodiments, a common data harvesting and transmission unit is arranged on the outside of a set of pods 100, e.g. at an office ceiling. The common data harvesting and transmission unit may harvest the measurement data from the pods 100, process the data, if required, and transmit further. The technical implementation of the cloud service 200 depends on the embodiment. In certain embodiments, there may be a data processing device or a server within the organization receiving and analyzing gathered data. And, the conversion into usable information may occur, depending on the embodiments, in various places (e.g. in the sensor itself, in a pod communication unit or similar, in a data harvesting and transmission unit or similar, at some user device, or at the network).

Depending on the size of the organization, the cloud service 200 may receive physiological data of a plurality of unidentified persons from a plurality of measurements points within the organization. The organization may contain a plurality of office pods similar to the preceding pod 100, which all provide the cloud service 200 with physiological measurement data. Depending on the size of the pods, each pod 100 may comprise a variable number of seats or sofa(s). Each seat or each seat of a sofa may comprise one or more non-wearable sensors for each seat.

Further, corresponding sensors 11/13/13′ may be provided at other meeting facilities of the organization, also for example at seats of conventional workstations (or workrooms), and/or in cafeterias or restaurants within the organization. A common feature of the gathered measurement data is that the measurement data is physiological data of a plurality of unidentified persons within the organization. The gathered data is anonymous aggregated data in that sense that the data is combined based on performed measurements on a plurality of unidentified persons. The gathered data is thus not linked to individual users (employees) of the organization. In a shared single user pod, different employees use the pod at different times. In larger pods (multi-user pods), more than one user may use the pod simultaneously.

The instant method further comprises detecting events or trends indicative of wellbeing from the gathered data, which will be described in more detail later in this description. Herein, by an event is meant an occurrence at which a single change in organizational wellbeing is detected. A stress peak or a day with a greater amount of laughter compared to an average may serve as examples of different events. Trends are long-term (e.g. monthly, yearly) indicators of general direction in which organizational wellbeing is developing or changing.

Next, turning to FIG. 4, a block diagram of an apparatus 40 according to an embodiment is shown. The apparatus 40 is for example a general-purpose computer or server or some other electronic data processing apparatus. In a specific embodiment, the general-purpose block diagram shown in FIG. 4 represents blocks of the sensor 11/13/13′, blocks of the pod communication unit 12, and blocks of a computer or an analyzing apparatus within the cloud service 200 as well. Accordingly, the apparatus 40 can be used for implementing at least some embodiments of the invention.

The apparatus 40 comprises a communication interface 45, a processor 41, a user interface 44, and a memory 42.

The communication interface 45 comprises in an embodiment a wired and/or wireless communication circuitry, such as Ethernet, USB, Wireless LAN or WI-FI, Bluetooth, GSM, CDMA, WCDMA, LTE, and/or 5G circuitry. The communication interface 45 can be integrated in the apparatus 40 or provided as a part of an adapter, card or the like, that is attachable to the apparatus 40. The communication interface 45 may support one or more different communication technologies. The apparatus 40 may also or alternatively comprise more than one communication interface 45.

The processor 41 may be a central processing unit (CPU), a microprocessor, a digital signal processor (DSP), a graphics processing unit, an application specific integrated circuit (ASIC), a field programmable gate array, a microcontroller or a combination of such elements.

The user interface 44 may comprise a circuitry for receiving input from a user of the apparatus 40, e.g., via a keyboard, graphical user interface shown on the display of the apparatus 40, speech recognition circuitry, microphone, or an accessory device, such as a headset, and for providing output to the user via, e.g., a graphical user interface or a loudspeaker.

The memory 42 comprises a work memory 43 and a persistent (non-volatile, N/V) memory 46 configured to store computer program code 47 and data 48. The memory 46 may comprise any one or more of: a read-only memory (ROM), a programmable read-only memory (PROM), an erasable programmable read-only memory (EPROM), a random-access memory (RAM), a flash memory, a data disk, an optical storage, a magnetic storage, a smart card, a solid state drive (SSD), or the like.

The apparatus 40 may comprise a plurality of memories 46. The memory 46 may be constructed as a part of the apparatus 40 or as an attachment to be inserted into a slot, port, or the like of the apparatus 40 by a user or by another person or by a robot. The memory 46 may serve the sole purpose of storing data, or be constructed as a part of an apparatus 40 serving other purposes, such as processing data.

In a sensor implementation of the apparatus 40, the apparatus further comprises the preceding sensor 11 and/or 13 and/or 13′. The processor 41 may then perform, as instructed by the program code 47, the initial processing of the measured physiological signal or data as provided by the sensor 11/13/13′. The communication interface 45 is used to transmit the measurement data further. The sensor 11/13/13′ may be implemented separately or as a part of the apparatus 40.

In a pod communication unit implementation of the apparatus 40, the communication interface 45 receives the measurement data from the sensor 11/13/13′, and the processor 41, if needed, and as instructed by the program code 47, performs processing of the received measurement data.

In an analyzing apparatus implementation of the apparatus 40, the communication interface 45 receives the physiological data of a plurality of unidentified persons from at least one non-wearable sensor. The received data is stored at the data section 48 of the apparatus 40. The processor 41, as instructed by the program code 47, performs analysis of the received measurement data to detect events or trends indicative of wellbeing from the received data. The detected events or trends can be shown on the user interface 44 and/or reported via the communication interface 45 e.g. to the accessing terminal device 300.

A skilled person appreciates that, depending on the embodiment, in addition to the elements shown in FIG. 4, the apparatus 40 may comprise other elements, such as further microphones, displays, as well as additional circuitry such as an input/output (I/O) circuitry, memory chips, application-specific integrated circuits (ASIC), a processing circuitry for specific purposes such as a source coding/decoding circuitry, a channel coding/decoding circuitry, a ciphering/deciphering circuitry, and the like. Additionally, the apparatus 40 may comprise a disposable or rechargeable battery (not shown) for powering the apparatus 40 when an external power supply is not available. Further, a microphone of the apparatus 40 may be used as a sensor to identify physiological data, especially laughter. In certain embodiments, this microphone represents the microphone comprised e.g. by the audio system 16 of the pod 100 discussed in the preceding with reference to FIG. 1.

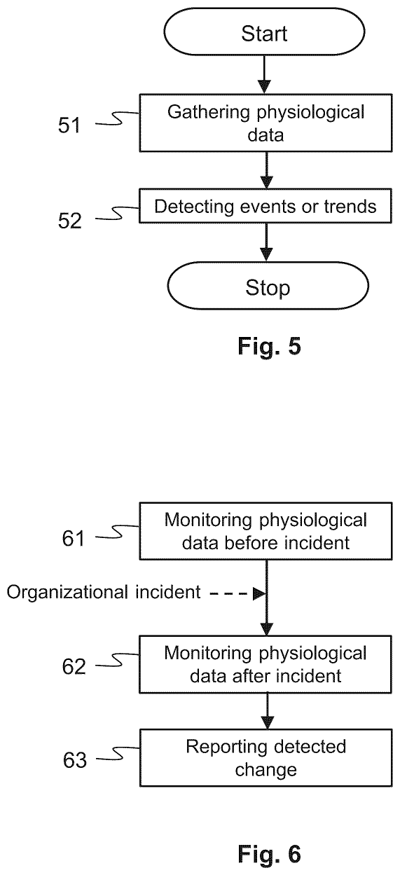

FIG. 5 shows a flow chart of a method in accordance with certain embodiments. In step 51, physiological data of a plurality of unidentified persons is gathered from at least one non-wearable sensor within an organization.

The physiological data is indicative of physiological response(s) to human emotional state(s) and/or indicative of human physical condition(s). In certain embodiments, the at least one non-wearable sensor is in conjunction with a meeting space, such as in an office pod as described. The sensor may be a seat-embedded sensor providing a BCG signal.

In step 52, events or trends indicative of organizational wellbeing is detected from the gathered data. This involves comparing physiological data of said plurality of unidentified persons at different points in time. In practice, it may be that the persons whose physiological data is being measured at a first time instant are not exactly the same persons whose physiological data is measured at a later second time instant. Accordingly, in certain embodiments, detecting the events and trends comprises comparing anonymous physiological data of a first set of persons (representing the population of the organization) with anonymous physiological data of a second set of persons (representing the population of the organization) over time (or between time instants), wherein the first and second sets of persons differ from each other. However, although the populations and/or the representative sets are not exactly the same, they are statistically comparable with each other, and reliable results can be obtained.

Laughter can be extracted from the raw sensor data by identifying discontinuities in a respiration cycle together with jiggling movements of the body. By monitoring the development of the amount of laughter within the organization, trends of organizational happiness are obtained.

Further, trends of physiological stress are detected by identifying heart rate variability (HRV) in the gathered data over time.

Further, trends of average physical condition of persons within the organization can be detected by monitoring heartbeat shapes over time.

In certain embodiments, the effect of personnel changes (for example, recruitments or persons leaving the organization) on organizational wellbeing is detected by monitoring indicators of negative bodily stress and/or organizational happiness.

An operator is provided with access (see e.g. FIG. 2, terminal 300) to the detected events and/or trends for appropriate organizational action to be taken. The detection of events and trends are based on averages (or based on other statistical measures) in physiological data due to the fact that the persons whose physiological data is analyzed are not identified for the purpose of the analysis.

FIG. 6 shows a flow chart of a method in accordance with certain other embodiments, relating to physiological response to a known organizational incident, for example an announcement of layoffs or a major organizational restructuring. In step 61, anonymous physiological data of a plurality of unidentified persons is gathered or monitored before the organizational incident. In step 62, physiological data of a plurality of unidentified persons is gathered or monitored after the organizational incident. A change in the physiological data is detected and reported to an operator in step 63. The change is based on averages (or based on other statistical measures) in physiological data due to the fact that the persons whose physiological data is analyzed are not identified for the purpose of the analysis.

FIGS. 7a-7d show identification of certain features in the case of a ballistocardiographic (BCG) signal in accordance with certain embodiments. FIG. 7a shows an illustrative example of the BCG signal 71 as received from a sensor. Heart rate variability can be calculated by applying as such well-known statistical analyses such as SDNN (the standard deviation of NN intervals) or RMSSD (root mean square of successive differences) for identified intervals d1, d2, d3, . . . of adjacent pulses, i.e. heartbeats, as illustrated in FIG. 7b. A respiration curve, as illustratively represented in the BCG signal 71, is shown in FIG. 7c. Laughter is identified e.g. as tightly-packed large-magnitude pulses in the BCG data as received from a sensor, as illustratively represented in the BCG signal 71′ in FIG. 7d. The reference numeral 75 illustrates a period of a short laugh, the reference numeral 76 a big laugh and the reference numeral 77 a very short laugh. Further, the reference numeral 78 in FIG. 7d illustrates a period of speaking aloud.

The disclosed embodiments provide a non-invasive way to measure the wellbeing of employees, particularly negative stress and organizational happiness, without the user wearing anything or attending to any additional devices or performing any overt actions.

Instead of a seat-embedded sensor, a floor-embedded or carpet-embedded sensor may be used in other embodiments suitable for measuring physiological data in scenarios involving persons standing on the floor/carpet while working.

In the event the office pods have more than one seat or multi-user seats, the number of disclosed sensors can be increased and/or a plurality of sensing zones can be included.

In addition to measuring physiological stress within the organization and/or organizational happiness, or alternatively, the disclosed sensor(s) are used to measure additional stress-related phenomena, such as restlessness (movement on the seat over time) and/or fatigue-related phenomena such as yawning (disruption in a normal breathing pattern) in certain embodiments. As an additional possibility, pressure-based seat sensors can be used to track organization-wide weight gain or loss, a well-known wellbeing indicator and determinant. In certain embodiments, the disclosed sensor(s) are used to detect (or measure) bodily restlessness (a negative phenomenon indicative of psychological uneasiness) and/or excessive bodily passivity (also a negative phenomenon indicative of lack of caring or engagement) e.g. in a group setting (e.g. a team meeting in a large booth).

Further, the seat-embedded sensor can serve a simultaneous function as a presence sensor in the pod (presence=detected sitting on a seat) and, in a multi-user setting, as an occupancy count sensor (number of people in a pod=detected sitters on seats).

Without limiting the scope and interpretation of the patent claims, certain technical effects of one or more of the example embodiments disclosed herein are listed in the following. A technical effect is an entirely invisible and non-detectable way to measure organizational wellbeing. An advantage of a seat-embedded ballistocardiographic measurement in an office pod is that it does not require any visible or potentially privacy-invading equipment (such as a camera) while measuring directly stress-linked data (such as heart rate variability) or laughter. The disclosed sensors are able to produce the measurement signals without the user having to wear any sensing device. Additionally, the user does not have to perform any actions (such as answer to a survey or a questionnaire or push any buttons), i.e., the user just uses the pod as usual and the sensor performs measurement(s) unnoticeably. Once the measurement(s) are carried out unnoticeably, the data coverage and unbiasness of the measurement(s) are improved. A further technical effect is the ability to provide collective measurement data (physiological data) without the users having to actively do anything except being present.

An advantage of certain embodiments is that they enable making inferences of organizational-level changes without measuring (or learning or storing) individual baselines for physiological signals. This is due to the fact that when measuring a statistically meaningful population and/or when measuring a relatively unchanging population, collective-level changes indicate changes in collective stress (or happiness, etc.). Accordingly, no establishment of personal baselines (meaning that an individual needs to be identified, too) before drawing conclusions about stress (or happiness, etc.) is needed.

Various embodiments have been presented. It should be appreciated that in this document, words “comprise”, “include”, and “contain” are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A method of measuring organizational wellbeing, comprising:
gathering physiological data of a plurality of unidentified persons from at least one non-wearable sensor within an organization; and
detecting events or trends indicative of organizational wellbeing from the gathered data.

2. The method of claim 1, wherein said physiological data is indicative of physiological response(s) to human emotional state(s) or indicative of human physical condition.

3. The method of claim 1, wherein said gathering physiological data comprises:
gathering physiological data of a plurality of unidentified persons from at least one non-wearable sensor in conjunction with a meeting space.

4. The method of claim 3, wherein said at least one non-wearable sensor is selected from a group consisting of sensors that detect movement, such as a pressure sensor, a radar, and/or an acceleration sensor.

5. The method of claim 3, wherein said at least one non-wearable sensor is integrated into a seat within the meeting space.

6. The method of claim 3, wherein the meeting space is a soundproof booth.

7. The method of claim 1, wherein said gathering physiological data comprises obtaining a ballistocardiographic (BCG) signal.

8. The method of claim 1, wherein said gathering physiological data comprises identifying laughter.

9. The method of claim 1, wherein said gathering physiological data comprises identifying an indication of physiological stress.

10. The method of claim 1, wherein said detecting events or trends comprises comparing gathered physiological data at an organizational level between different points in time.

11. The method of claim 1, wherein said detecting events or trends comprises detecting an instant statistical change and/or a long-term statistical change in gathered physiological data.

12. The method of claim 1, wherein said detecting events or trends comprises:
monitoring physiological data before and after an organizational incident; and
reporting a detected statistical change in said physiological data after the incident.

13. An apparatus, comprising:
at least one processor; and
at least one memory including computer program code, the at least one memory and the computer program code being configured, with the at least one processor, to cause the apparatus to perform the method of claim 1.

14. A computer program comprising computer executable program code which when executed by at least one processor causes an apparatus to perform the method of claim 1.

15. A soundproof booth for use in the method of claim 1, comprising:
a non-wearable sensor for gathering physiological data of a plurality of unidentified persons within the booth for an apparatus that gathers physiological data of a plurality of unidentified persons from at least one non-wearable sensor within an organization and detects events or trends indicative of organizational wellbeing from the gathered data.
